(19) United States
(12) Patent Application Publication
(10) Pub. No.: US 2008/0318342 A1
(43) Pub. Date: Dec. 25, 2008

(54) Diagnostic Testing Process and Apparatus Incorporating Controlled Sample Flow

(76) Inventors: Matthew C. A. Durack, New South Wales (AU); Rowan Buls, New South Wales (AU)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/628,605

(22) PCT Filed: Jun. 6, 2005
(86) PCT No.: PCT/AU2005/000797

§ 371 Date: Mar. 21, 2008

(30) Foreign Application Priority Data
Jun. 4, 2004 (AU) 2004903009

(51) Int. Cl. G01N 33/553; B01J 19/00; G01N 33/543

(52) U.S. Cl. 436/526; 422/68.1; 436/518

(57) ABSTRACT

An apparatus (10) and method for use in a vertical flow-through assay process is characterised by applying pressure to a sample to force the sample through a reaction/capture membrane (12), to which one or more ligands are bound, at a controlled rate. Typically, the method includes a pre-incubation step in which the sample and a detection analyte typically an antibody bound to colloidal gold or a fluorescent tag bind together The pre-incubation step typically takes place in a chamber (26) spaced above the capture membrane (12). The base of the chamber is defined by a porous hydrophobic frit (34) typically formed from polyethylene. It is preferred that the chamber (26) is defined by the upper part of a cylinder (24) extending from a seal (30) compressing the reaction membrane (12) against an absorbent pad (28). The seal (30) has the effect of compressing tie reaction membrane and preventing wicking of the sample in the lateral direction outside of the circular seal. A piston (28) compresses air located in the chamber above atmospheric pressure to farce the sample to pass through the hydrophobic frit (34). Alternatively, a hydraulic actuator (60) may directly act on the sample to force the sample through the frit and reaction membrane at a predetermined rate.

### CROSS-REFERENCE TO RELATED APPLICATIONS

The present application claims priority from Provisional patent application Ser. No. 2004903009 filed on 4 Jun. 2004, the content of which is incorporated herein by reference,

### FIELD OF THE INVENTION

This invention relates to a diagnostic testing process and apparatus. In particular, the invention relates to an apparatus for use in carrying out an assay process incorporating controlled flow of the sample being assayed and to a method of carrying out all assay process incorporating controlled flow.

### BACKGROUND OF THE INVENTION

Lateral flow and flow-through technology have been used for diagnostic assays for almost twenty years. Lateral flow technology is currently dominant because lateral flow devices are easy to produce and the assay can be performed in a simple two step process that can be adapted for whole blood separation. This results in a simple device that can be used in the field as a rapid point-of care diagnostic (see Cole et al 1996 Tuberc. Lung. Dis. 77:363-368). However, multiple disease diagnosis using lateral flow technology is very difficult because of differences in lateral diffusion between samples and variation in flow rates between batches of the partitioning membranes. This means that antigen or antibody signal strengths may vary both within tests and between batches of tests, resulting in inconsistent results.

Flow-through diagnostic tests can be completed in less than two minutes compared with typical times of five to fifteen minutes for lateral flow tests. This advantage in speed however, is often at the expense of sensitivity.

The basic principle of flow-through assays is well established. The tests are designed to determine the existence of, and in some cases the quantity of a predetermined analyte/reagent in a sample. Often, the reagent will be a protein, but other reagents can be tested for. If the assay is the test for the existence of a particular disease in a patient, the patient's body fluids may be tested for an antibody or other protein produced by the patient in response to the infection, or for a protein which is expressed by the bacterium or viral agent or the like causing the disease. In a typical flow-through assay a liquid sample, which is believed to contain the reagent, is sucked into an absorbent pad via a membrane which is bound to capture analyte which is known to bind to the reagent. The membrane is then typically washed with a buffer. A liquid containing a detection analyte which also binds to the reagent and which includes a tracer or marker which is detectable, is applied to the membrane. The detection analyte binds to the immobilised reagent bound to the membrane and can be seen or otherwise detected to indicate the presence of the reagent.

International Patent Application No. PCT/AU02/01119 discloses an improved flow-trough apparatus for use in an assay process which is characterised by providing a pre-incubation step in which the sample and detection analyte bind together prior to flow through of the sample and analyte onto the capture membrane. This apparatus has improved sensitivity over existing vertical flow through apparatus and a further advantage is that a reduced the volume of sample is required for an assay. It has a further advantage that it is possible to analyse larger sample volumes (e.g. mls of blood) hence it is possible to detect reagents at low concentrations. However, although the apparatus and method disclosed in PCT/AU02/01119 is an improvement over existing vertical flow-through devices it has been found that considerable variation in the results occurs. This variation is thought to be due to variations in the characteristics and porosity of the (typically nitrocellulose) membranes to which the capture analyte is bound.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application,

Because the prior art is not consistent in its terminology, for the avoidance of doubt and for the purpose of clarity the following terms used in the specification below, are defined as follows. The term “reagent” is used to refer to the compound, protein or the Mm which is to be detected by the assay. The term “capture analyte” is used to refer to a compound which is bound to a membrane and to which the reagent will bind. The term “detection analyte” is used to refer to a compound which will also bind to the reagent and which carries a tracer or some other element whose presence may be detected, typically visually detected whether under visible light or fluorescence.

### SUMMARY OF THE INVENTION

In a first broad aspect, the present invention provides an apparatus and method for use in a vertical flow-through assay process which is characterised by applying pressure to a sample to force the sample at a controlled rate through a reaction/capture membrane to which one or more ligands are bound. It is preferred that the method includes a pre-incubation step in which the sample and detection analyte typically an antibody bound to colloidal gold or a fluorescent tag bind together.

In more detail, in one aspect of the present invention there is provided an apparatus for use in an assay process comprising:

a porous reaction membrane to which is bound capture analyte for binding to a reagent to be detected, the membrane having an upper surface and a lower surface;

a body of absorbent material such as tissue paper or the like disposed below and touching the lower surface of the reaction membrane;

a chamber spaced above the first member said chamber having side walls, and a base defined by a second membrane, the chamber being supported generally vertically above the first member and characterised by means for forcing liquid sample contained in the chamber under pressure through the base of the chamber and onto the reaction membrane.

In one preferred embodiment the base of the member is defined by a porous hydrophobic frit typically formed from polyethylene.

The chamber may be defined by the upper part of a cylinder extending from a seal compressing the reaction membrane against the absorbent pad. The seal has the effect of compressing the reaction membrane and preventing wicking of the sample in the lateral direction outside of the circular seal.

In one embodiment the means for pressuring the sample may include a piston means which can be used to compress gas, typically air located in the chamber above to pressurising the chamber and force the sample to pass through the hydrophobic flit

In another embodiment the means for forcing the sample through the base of the chamber may comprise a hydraulic actuator directly acting on the sample forcing the sample through the flit and reaction membrane at a predetermined rate.

The absorbent body and reaction membrane may be located in a housing which defines at least one bleed aperture to prevent the build up of pressure inside the casing.

In one particular preferred embodiment a plurality of such apparati are linked together and pistons are used to force the sample through the frits onto the membranes simultaneously and at the same rate.

### BRIEF DESCRIPTION OF THE DRAWINGS

A preferred embodiment of the present invention will now be described by way of example only with reference to the accompanying drawings:

FIG. 1 shows a section through a first diagnostic test apparatus;

FIG. 2 is a perspective view of the apparatus shown in FIG. 1;

FIG. 3 shows the apparatus of FIG. 1 in use during a pre-incubation step;

FIG. 4 shows the apparatus of FIG. 1 with a plunger cup inserted;

FIG. 5 shows the apparatus of FIG. 1 after a sample has flowed through a nitrocellulose membrane of the apparatus;

FIG. 6 shows a plunger and cylinder assembly removed from the apparatus for washing of the membrane and reading of the results;

FIG. 7 shows a second embodiment of a diagnostic testing device incorporating a hydraulic flow through control;

FIG. 8 is a perspective view of the apparatus of FIG. 7;

FIG. 9 shows the apparatus of FIG. 7 in use at pre-incubation stage;

FIGS. 10 and 11 illustrate flow-through of the sample in the apparatus of FIG. 7;

FIGS. 12 and 13 illustrate the removal of the screw drive actuator and cylinder and piston respectively from the apparatus for the reading of a result;

FIG. 14 shows a cross-section through a third embodiment of a diagnostic testing apparatus comprising a plurality of diagnostic tests and

FIG. 15 is a perspective view of the apparatus shown in FIG. 14.

### DETAILED DESCRIPTION OF THE PREFERRED EMBODIMENT

Capture analytes in the form of ligands such as antigens or antibodies are printed onto a protein capture membrane matrix, more particularly, a nitrocellulose membrane in an appropriately sized array using piezoelectric chemical printing technology or other printing technologies, such as syringe pump. A suitable chemical printing system involves the use of piezoelectric drop on demand inkjet printing technology for micro dispensing fluids, in DNA diagnostics or, a Combion Inc. synthesis process, called “CHEM-JET”. Such a device including an imaging means is also described in the Applicant's International Patent Application No. PCT/AU98/00265, the entire contents of which are incorporated herein by reference. In the described embodiment, antigen is printed onto a reactions membrane in 100 PL droplets, or multiples thereof with each aliquot being 1 mm apart. However, these volumes and distances can be increased/decreased accordingly depending on the chosen antigen titre and array size.

In a particular embodiment antigens or antibodies are printed down onto a nitrocellulose membrane having a pore size of 0.22 microns in a matrix of dots to form lines. After the dispensed antigen has dried, non-specific protein binding sites on the nitrocellulose membrane are blocked through use of a buffer that blocks available sites on the nitrocellulose membrane.

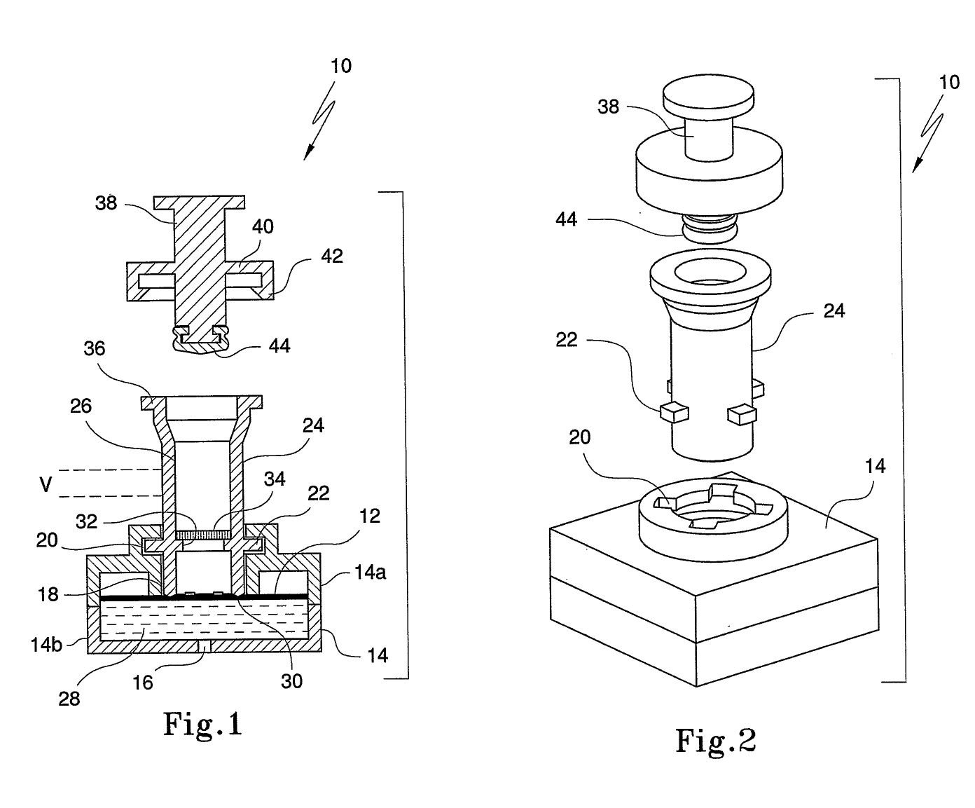

Turning now to the drawings, FIG. 1 shows a flow-trough assay device 10 which utilises the nitrocellulose membrane 12 described above. The device 10 includes a cassette or housing 14 which is made in two halves, an upper half 14a and a lower half 14b. A bleed hole 16 is defined in the base of the lower half 14b. the upper half 14a of the casing defines a cylindrical aperture 18 at the base of a well, the sides of which define recesses 20 for receiving bayonet fittings 22 defined on a generally cylindrical insert 24 which inter alia, defines a pre-incubation chamber 26.

The nitrocellulose insert is located inside the casing at the base of the insert 24 and on top of an absorbent matrix 28. The absorbent matrix typically comprises multiple layers of absorbent tissue or an absorbent pad such as a blotting paper.

As can be seen from FIG. 1, the base 30 of the insert 24 presses down on the membrane 12 and acts as a seal 30 to inhibit lateral wicking of sample in the membrane 12 past the seal. A flange 32 extends around the interior of the insert 24 close to its base which supports a porous hydrophobic polyethylene frit 34 which defines the base of the pre-incubation chamber. The pore size of the frit 34 is 10 to 200 microns, which is approximately one hundred to one thousand times the pore size of the membrane 12. The upper end of the insert 24 defines an external flange 36.

A cylindrical piston/plunger 38 is provided having the same external diameter as the internal diameter of the pre-incubation chamber. The top of the plunger defines an external flange 40 from which a snap fit mechanism 42 depends. A seal 44 is defined at the bottom of the plunger.

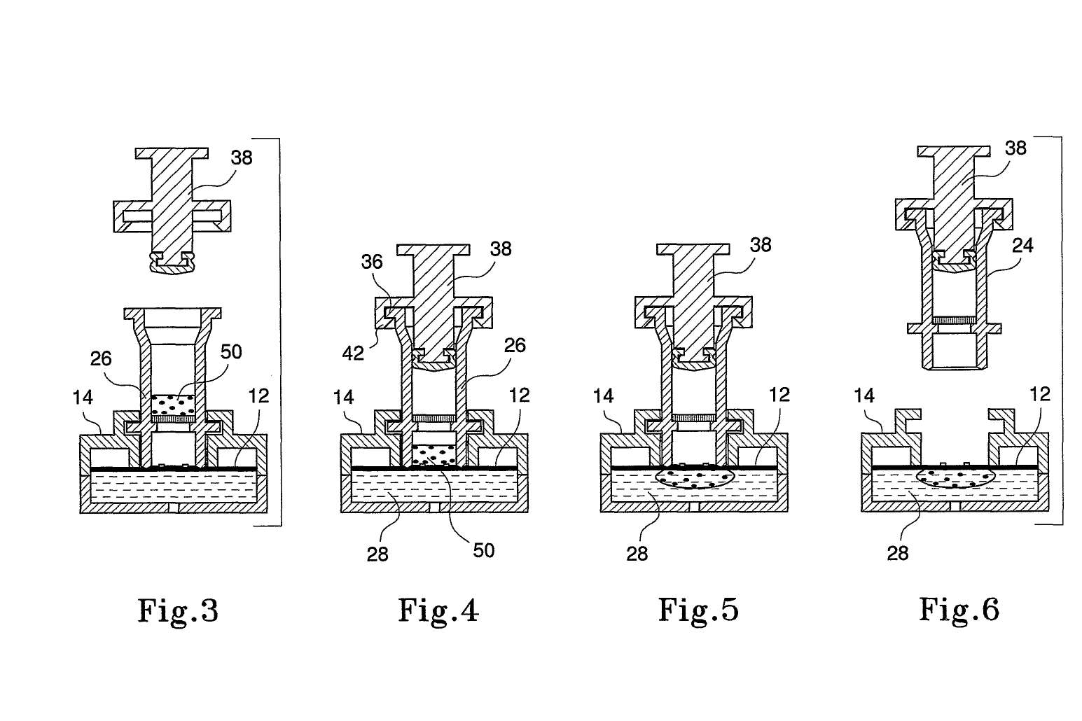

In use, with reference to FIGS. 3 to 6, a sample 50 to be assayed is placed in the pre-incubation chamber 26 as shown in FIG. 3 together with a detection analyte. The sample volume is typically approximately 1.5 to 2 ml. Because the frit is hydrophobic the solution does not penetrate the frit and remains in the incubation chamber. A predetermined period of time is allowed for pre-incubation, typically for 30 seconds.

Next, as shown in FIG. 4, the plunger is inserted into the open end of the cylindrical pre-incubation chamber 26 and the external flange of the piston, pushed down until the snap fit mechanism 42 snaps fit behind the flange 36 at the top of the pre-incubation chamber. No further movement of the piston takes place. The piston compresses a predetermined volume “V” of air inside the pre-incubation chamber pressurising the sample to force it to flow through the frit 34 onto the membrane 12, as shown in FIG. 4.

The sample, which is still under increased (above atmospheric) pressure then flows through the nitrocellulose membrane 12 as shown in FIG. 5. The increased pressure is believed to improve the results since a sample is driven through the nitrocellulose membrane by the pressure more quickly and evenly than it would ordinarily wick through under gravity. The compression of a predetermined volume of air and allowing the sample to flow through under that pressure, improves the accuracy and consistency of the diagnostic test. The sample will flow quickly through the frit in approximately 10 seconds and then more slowly through the nitrocellulose membrane, typically taking approximately 50 seconds. The flow time is generally consistent even with membranes from different batches which may have different hydrophobicity and pore size. Contact between the pad 28 and the membrane 12 is improved by the increase in pressure and this is thought also to be a factor in improving the reproducibility of the apparatus.

Next, as shown in FIG. 6 the pre-incubation chamber 24 and piston 38 are removed as one. Next, two or three drops of wash solution are applied to the nitrocellulose membrane 12 and the result is then interpreted in a reader (not shown).

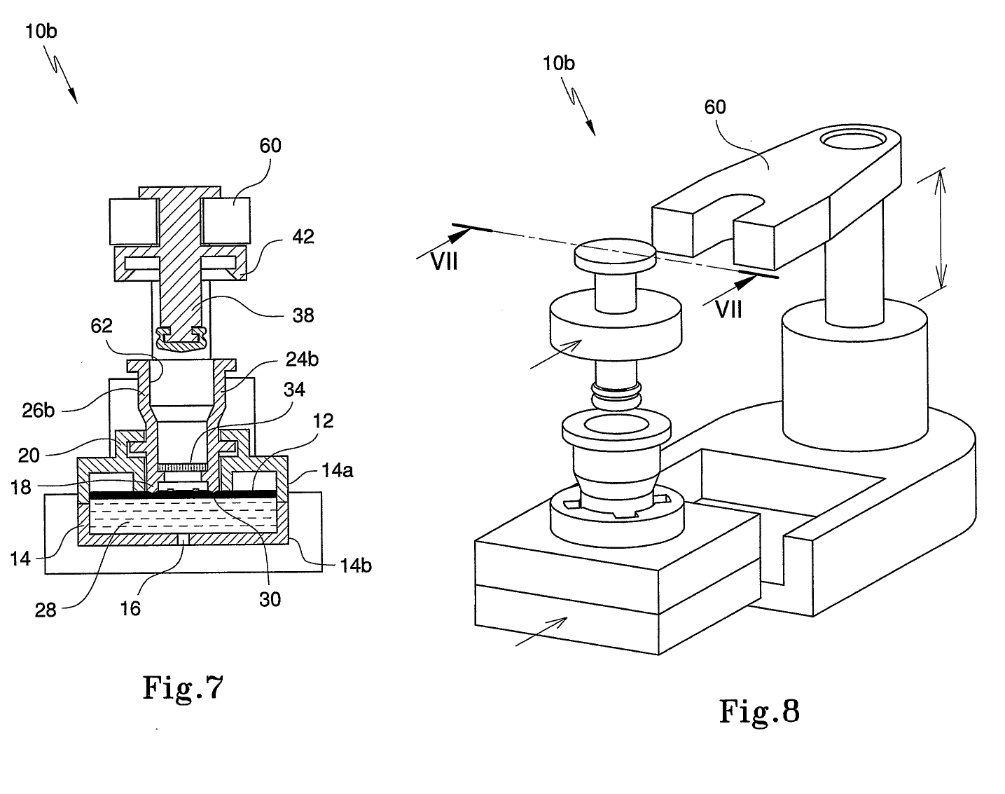

FIG. 7 to 13 illustrate a second embodiment of a vertical flow through assay apparatus 10b in which the control flow-through is achieved by means of a hydraulic control device in the form of a screw drive actuator 60. The design of the apparatus is very similar to that of the embodiment shown in FIGS. 1 to 6, utilising many of the same components, and identical components which carry the same reference numerals, will not be described in detail.

In the second embodiment the shape of the cylindrical insert 24b/pre-incubation chamber 26b is slightly different in design from that of the first embodiment In particular, the upper part 62 of the insert is relatively wider than the lower part. It is also relatively wider than the diameter of the piston 38. In this embodiment the sample is pushed through using the screw drive actuator at a predetermined flow rate.

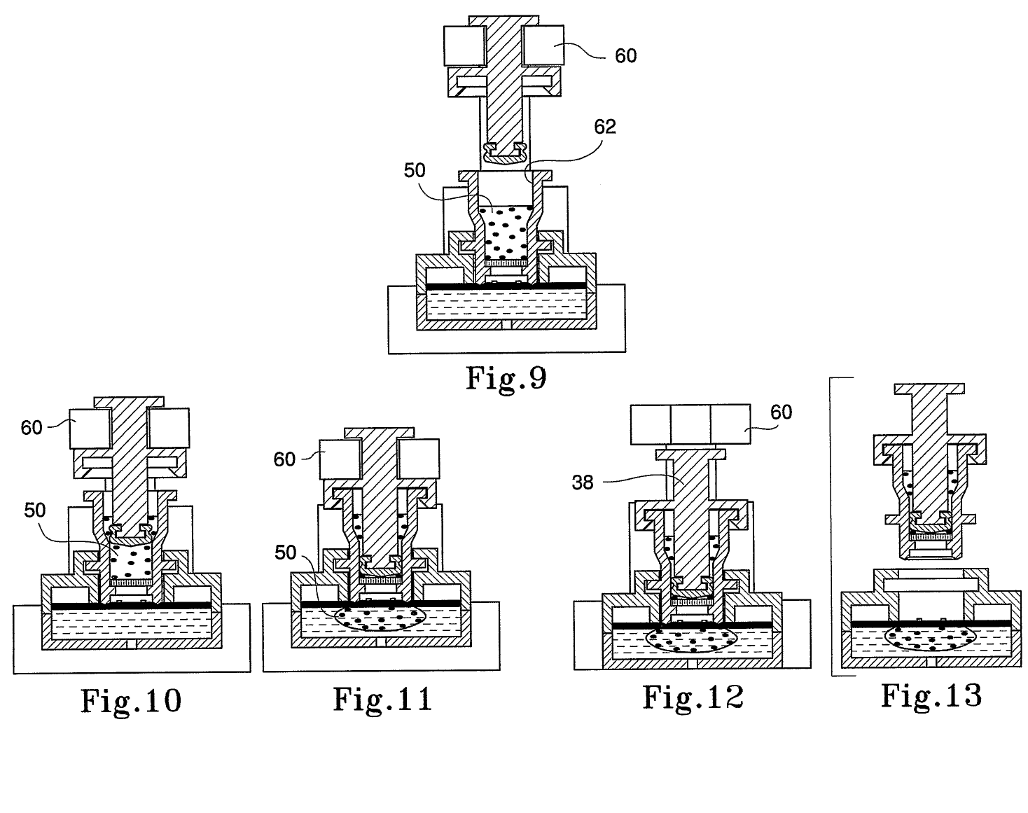

FIGS. 9 to 13 illustrate the use of the diagnostic test using the hydraulic control device 60. In particular, FIG. 9 shows the sample 50 and detection analyte (conjugate) are loaded into the pre-incubation chamber 26b to a level above the wider portion 62 of the pre-incubation chamber.

FIG. 10 shows the piston pressed into the pre-incubation chamber under the action of the screw drive actuator 60 which is set to move downwards at a predetermined rate so that a known flow rate of sample through the frit and nitrocellulose membrane takes place. Typically, the preferred flow rate is 90 ml per hour which equates to a 1.5 ml sample flowing through the fret and nitrocellulose membrane in approximately 1 minute. Loading the sample at least to the wider portion 62 of the pre-incubation chamber prevents air bubbles, although some sample is wasted. For quantitative analyses, a precise volume of sample is loaded, such that there is no sample wastage. FIG. 11 illustrates the situation after the sample has flowed through the frit 34 and membrane 12

FIG. 12 illustrates the removal of the screw drive actuator 60. FIG. 13 shows the subsequent removal of the pre-incubation chamber 26b and plunger 38, so that the nitrocellulose membrane 12 can be washed and the result read.

It has been found that by using the controlled flow through diagnostic test described above, greatly improved results are achieved with improved reproducibility and much greater consistency in tests utilising membranes from different batches. Nitrocellulose membranes may vary quite considerably in their hydrophobicity and pore size and with a simple vertical flow through test without controlled flow this has been found to have a considerable effect on the quality (such as the reproducibility) of the results.

A number of features are believed to contribute to the improved results including the fact that the pressure provides greater contact between the nitrocellulose membrane and the absorbent pad and reduces the effect of lateral wicking. The seal 30 also confines the sample to flowing vertically down through the wick The pressure has also been found to overcome the variations in pore size of the nitrocellulose membrane. The use of a piston/plunger also prevents splashback of sample.

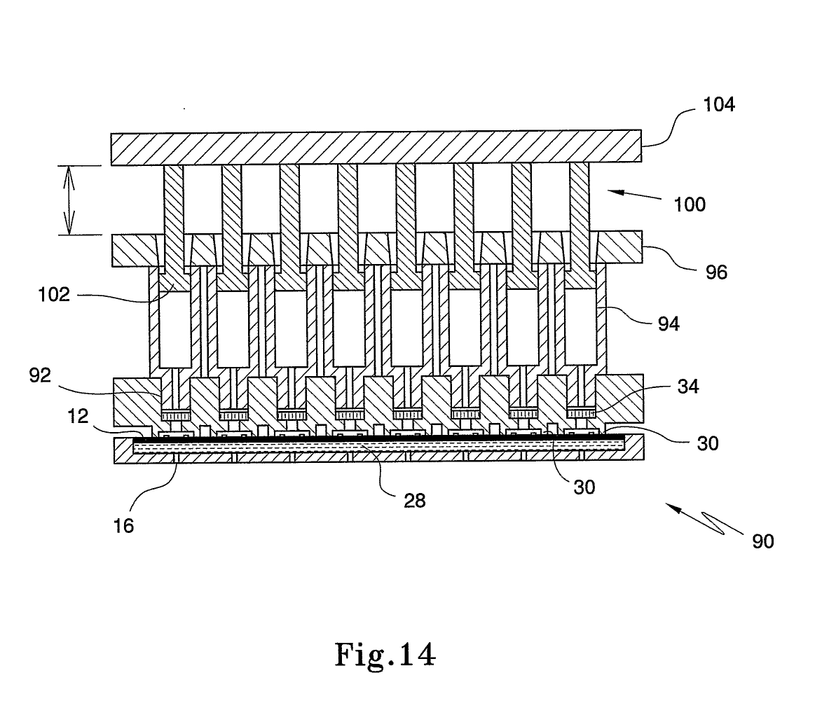

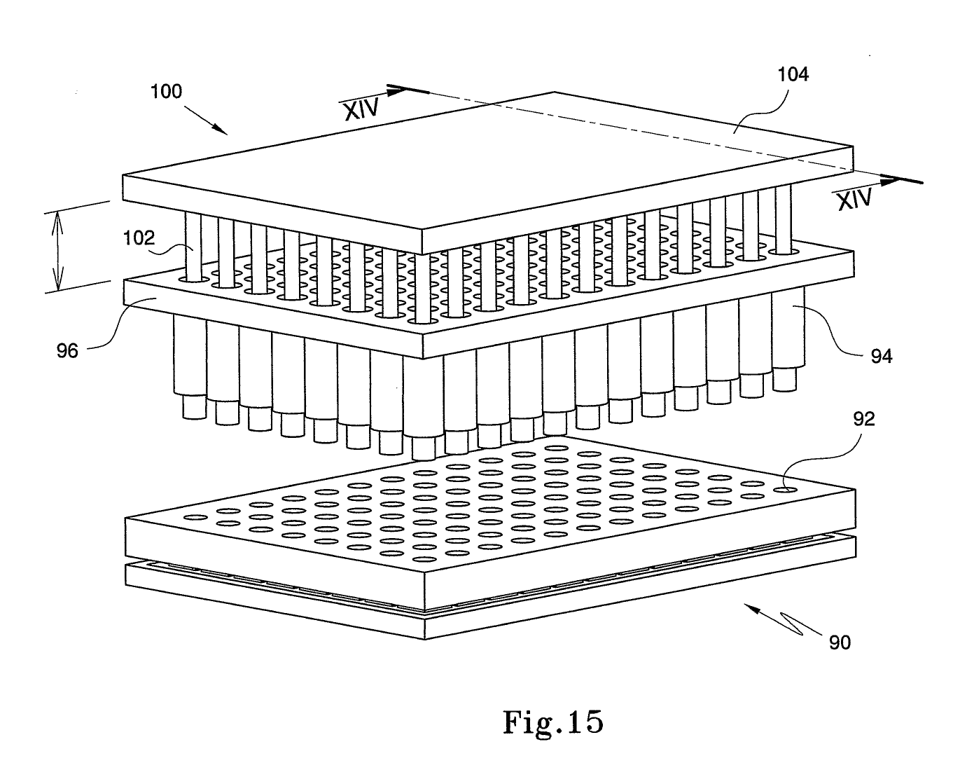

FIG. 14 illustrates a further embodiment of the present invention in which in the form of a 12 by 8 array of ninety six diagnostic test devices similar to the device of FIGS. 1 to 6 is disclosed and which operates in the same way.

The apparatus 90 uses a single nitrocellulose membrane 12 and a single blotting paper wick 28. There is an array of ninety six wells (12 by 8) 92 defined in the apparatus. Annular seals 30 are defined at the base of each well 92, as in the embodiment of FIGS. 1 to 6, and these prevent cross-contamination between adjacent samples. An array of ninety six cylindrical pre-incubation chambers 94 corresponding to the ninety six wells are mounted to a support plate 96 and are also arranged in a 12 by 8 array. The base of each chamber is again defined by a porous frit 34 There is a corresponding array 100 of ninety six pistons 102 attached to a support plate 104 which locate in the pre-incubation chambers 94 in the apparatus 90 and pressurise the sample. Using this apparatus all ninety six diagnostic tests can be run simultaneously, with pre-incubation done prior to depressing the pistons. The significant advantage of this system is that the membrane and the striping of the reagents of the membrane is consistent across the tests and the only variable in the process is the sample. Thus not only is the test quick to perform but also very consistent results can be expected.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An apparatus for use in an assay process comprising:
a porous reaction membrane to which is bound capture analyte for binding to a reagent to be detected, the membrane having an upper surface and a lower surface;
a body of absorbent material disposed below and touching the lower surface of the reaction membrane; and
a chamber for receiving a liquid sample spaced above the first member, said chamber having side walls, and a base defined by a second membrane, the chamber being supported generally vertically above the first member,
wherein, when in use, the apparatus further comprises means for forcing liquid sample contained in the chamber under pressure through the base of the chamber and onto the reaction membrane.

2. An apparatus as claimed in claim 1 wherein the second membrane comprises a porous hydrophobic frit.

3. An apparatus as claimed in claim 2 wherein the porous hydrophobic frit is formed from polyethylene.

4. An apparatus as claimed in claim 1 wherein the porous reaction membrane and body of absorbent material are held in a housing, an upper surface of which defines an aperture for insertion of the chamber, wherein the apparatus comprises means for releasably securing the chamber to the housing.

5. An apparatus as claimed in claim 4 wherein the chamber is defined by the upper part of a cylinder having an annular base is arranged to compress the reaction membrane against the absorbent pad within the housing thereby defining an annular seal for preventing wicking of the sample in a lateral direction outside of the circular seal.

6. An apparatus as claimed in claim 5 wherein the means for forcing the liquid sample comprises a pneumatic means.

7. An apparatus as claimed in claim 1 wherein the means for forcing the liquid sample comprise a piston means receivable in the chamber for compressing gas in the chamber to a pressure above atmospheric pressure for forcing the sample to pass through the second membrane under pressure.

8. An apparatus as claimed in claim 1 wherein the means for forcing the liquid sample comprise a hydraulic means.

9. An apparatus as claimed in claim 1 wherein the means for forcing the liquid sample comprise a hydraulic actuator directly acting on the sample volume and forcing the sample through the second membrane at a predetermined rate defined by the actuator.

10. An apparatus as claimed in claim 4 wherein the housing defines at least one bleed aperture to prevent the build up of pressure inside the casing.

11. An apparatus as claimed in claim 1 wherein multiple different capture analytes are bound to the reaction membrane.

12. An apparatus as claimed in claim 11 wherein the multiple different capture analytes are present as stripes or spots.

13. A diagnostic test apparatus comprising an array of a plurality of apparatuses as claimed in claim 7 linked together, where the piston means are linked to force samples through the second membranes of each chamber in the array simultaneously.

14. A diagnostic test apparatus as claimed in claim 13 wherein a single reaction membrane is disposed below the second membranes of a number of, or all of, the chambers in the array.

15. A method of carrying out a vertical flow-through assay process comprising the steps of:
placing a sample in a chamber having a base defined by a membrane, the membrane being spaced above a porous reaction membrane to which is bound to capture analyte for binding to a reagent to be detected in the sample, disposed below and touching the reaction membrane;
applying pressure to a sample to force the sample through the reaction membrane at a controlled rate.

16. A method as claimed in claim 15 including a pre-incubation step in which the sample and capture analyte are put together in the chamber for a predetermined period prior to application of pressure.

17. A method as claimed in claim 15 wherein the step of applying pressure includes compressing a predetermined volume of air located in the chamber above the sample.

18. A method as claimed in claim 15 wherein the step of applying pressure to a sample is carried out pneumatically.

19. A method as claimed in claim 15 wherein the step of applying pressure to a sample is carried out hydraulically.

20. A method of carrying out a vertical flow-through assay process as claimed in claim 15 wherein multiple chambers are provided above the base and multiple assays are carried out simultaneously.

21. A method of carrying out a vertical flow-through assay process as claimed in claim 15 wherein multiple different capture analytes are bound to the reaction membrane for testing for a plurality of different reagents simultaneously.

22. A method of carrying out a vertical flow-through assay process as claimed in claim 16 wherein the pre-incubation step has a duration of at least about 30 seconds.

23. A method of carrying out a vertical flow-through assay process as claimed in claim 15 wherein the sample has a volume of about 1.5 to 2 ml.

24. The method of claim 16, wherein the capture analyte is an antibody.

25. The method of claim 24, wherein the antibody is bound to colloidal gold or to a fluorescent tag.
